(19) United States
(12) Patent Application Publication
(10) Pub. No.: US 2001/0036671 A1
(43) Pub. Date: Nov. 1, 2001

(54) Optical antioxidant sensing process

(76) Inventor: Gina Lynn Nick, Rancho Santa Margarita, CA (US)

Correspondence Address: Gina L. Nick, PO Box 3156, Laguna Hills, CA, 92654, US

(21) Appl. No.: 09/814,151

(22) Filed: Mar. 21, 2001

Related U.S. Application Data
(60) Provisional application No. 60/192,251, filed on Mar. 25, 2000.

(51) Int. Cl. C12Q 1/28; G01N 21/75

(52) U.S. Cl. 436/164; 435/28

(57) ABSTRACT

A process for assaying the free radical scavenging capability of nutritional formulations using an organic dye reagent and an optical fiber sensor.

### CROSS REFERENCE TO RELATED APPLICATIONS

The present application claims the benefit of priority of Provisional Patent Application serial No. 60/192,251, filed Mar. 25, 2000 for Gina Lynn Nick the entire content of which is incorporated herein by reference.

### FIELD OF THE INVENTION

The present invention relates to a method of measuring the free radical scavenging capability of functional food-based source antioxidants.

### BACKGROUND OF THE INVENTION

It is generally believed that cellular damage caused by free radicals, particularly oxygen-radicals, is one of the major causes of disease in living organisms.

In the biological and medical arts, it is well known that the tissues, or cells of living tissue, have specific mechanisms to defend against free radicals, and that in some disease states these mechanisms are disturbed. As described in U.S. Pat. No. 5,395,755, for example, it is postulated that blood, including both cells and plasma, has a free-radical defense mechanism. The mechanism is recognized as complex involving substances capable of “scavenging” to give a total “anti-oxidant” property to a cellular system.

An abundance of nutritional supplement formulations having anti-oxidant properties are currently available and many claims are made about the effectiveness of a formulation because of its superior anti-oxidant properties.

Accordingly, there is a need to qualitatively and quantitatively assess the antioxidant capacity of foods and botanicals and other nutritional anti-oxidant supplements.

At the present time, a limited number of objective methods are available to measure the antioxidant capability of nutritional formulations. Methods and procedures that quantify the free-radical scavenging capability of specific formulations are known in the art and anti-oxidant capacity assays such as the total peroxyl radical trapping parameter (TRAP) assay, the Trolox equivalent antioxidant capacity (TEAC) assay, and the oxygen radical absorbance capacity (ORAC) assay have been used as a basis to compare and standardize nutritional supplements.

Each of these methods characterizes the end product of endogenous anti-oxidant activity in biological fluids, such as serum, to identify isolated anti-oxidants. These methods, however, do not measure the anti-oxidant capacity of specific cells.

### BRIEF SUMMARY OF THE INVENTION

The effectiveness of a nutritional formula can be characterized by measuring the protective effect offered by antioxidants in the formula and specifically the intracellular free radical scavenging capability of the functional food-based source antioxidants present in the formula.

It is a known feature of antioxidants that they will depress fluorescence observed from a number of different fluorescent specific sources. This property has been used with assays in an attempt to determine which radicals are preferably “mopped-up” by which antioxidant and to compare the efficiency of different food antioxidants.

It has been found that when a test sample is added to a progressing fluorescent reaction, the antioxidants present in the sample cause a transient reduction in the observed fluorescence (measured light output level). The level recovers close to its initial value after a short time. This time, during which the observed fluorescence is reduced, provides an index as to the capacity of the antioxidants in the test sample. Thus, the antioxidant capacity of a given sample can be assayed utilizing a fluorescent reaction. The term “assay” as used herein, includes qualitative, semi-quantitative, and quantitative indications, assessments, estimations, and measurements.

As indicated above, the Oxygen Radical Absorption Capacity (ORAC) assay is one method of measuring total serum antioxidant activity. This method quantitatively measures the total antioxidant capacity of a serum using a free radical sensitive fluorescent indicator protein phycoerythrin (beta-PE) to monitor the effectiveness of various serum antioxidants in protecting the beta-PE from becoming damaged by free radicals. Assay results are quantified by allowing the reaction to reach completion and then integrating the area under the kinetic curve relative to a blank reaction containing no antioxidant protection. The area under the curve is proportional to the concentration of all of the antioxidants present in the sample.

The unique optical antioxidant sensing process (OASP) of the invention assays the free radical scavenging capability of specific antioxidant fortified cells, when encountering various reactive oxygen species (ROS), by quantifying the intracellular chemical changes caused by oxidation, or loss of hydrogen groups. The invention uses a fluorescent dye probe and an optical fiber sensor to detect chemical changes in the dye.

### BRIEF DESCRIPTION OF THE SEVERAL VIEWS OF THE DRAWING

FIG. 1 is a functional flow chart of the OASP of the invention;

FIG. 2 is a detailed flow chart of the OASP of the invention;

FIG. 3 is a detailed flow chart of the comparative OASP of the invention;

FIG. 4 is a functional diagram of an in-vitro model of a gastrointestinal system using the OASP of the invention.

### DETAILED DESCRIPTION OF THE INVENTION

In accordance with the principles of the invention, an optical antioxidant sensing process (OASP) is used to evaluate the effectiveness of a given nutritional formulation.

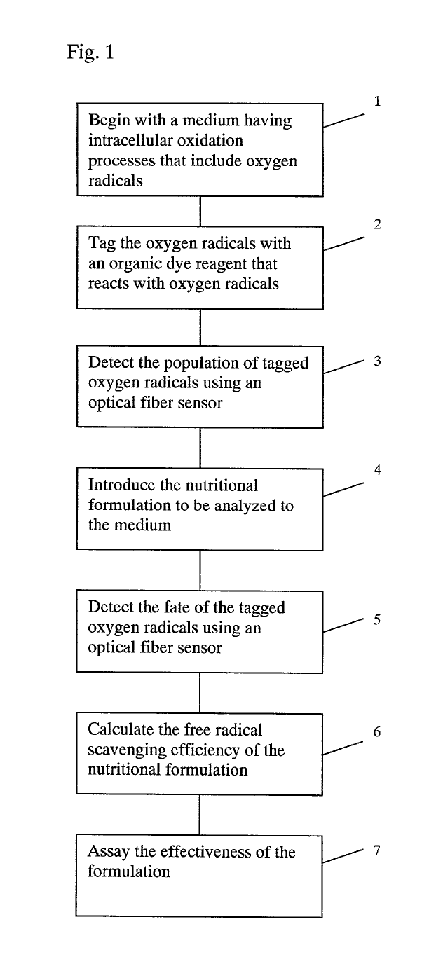

The process begins with a medium having intracellular oxidation processes that include oxygen radicals as shown in step 1 of FIG. 1. An organic dye reagent that reacts with oxygen radicals is introduced to the medium causing the oxygen radicals to be tagged by the dye as shown in step 2 of FIG. 1. The chemical composition of the reagent preferably mimics the composition of a human cell and is non-polar, by nature of its diacetate ester groups, which easily penetrates the plasma membrane.

The population of tagged oxygen radicals is measured using an optical fiber sensor as shown in step 3 of FIG. 1. Typically, the sensor transmits light at a wavelength of 488 nm, which excites electrons in the medium, to produce a characteristic fluorescence at a wavelength of 525 nm that is measured by the sensor.

The nutritional formulation to be analyzed is then introduced to the medium as shown in step 4 of FIG. 1 and the fate of the tagged oxygen radicals is detected using an optical fiber sensor as shown in step 5 of FIG. 1.

The free radical scavenging efficiency of the nutritional formulation is then calculated as shown in step 6 of FIG. 1 and an assay is made of the comparative effectiveness of the formulation as shown in step 7 of FIG. 1.

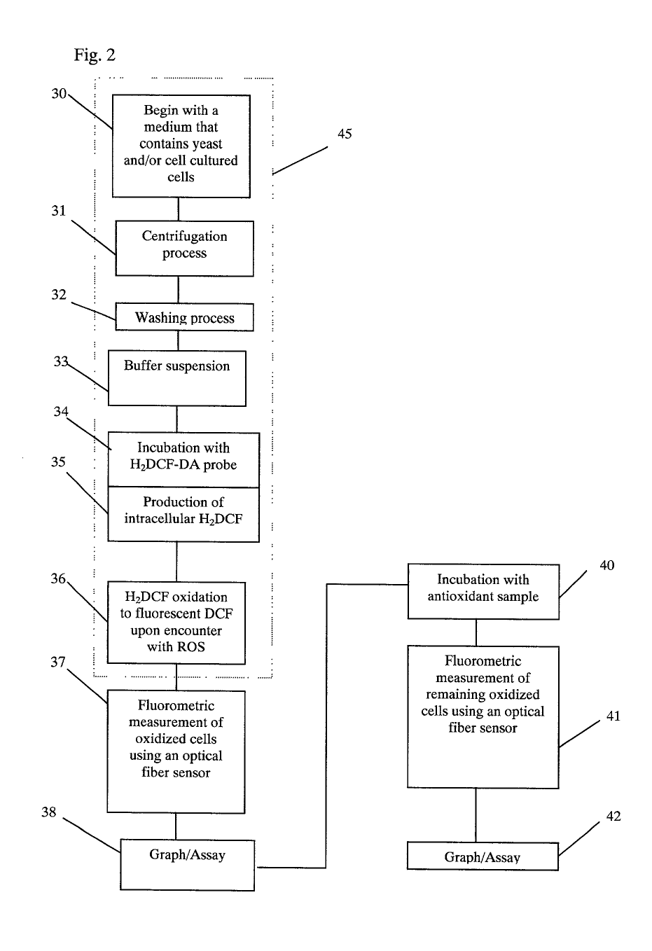

With reference to FIG. 2, the medium used in the OASP of the invention contains live yeast cells such as sacchromyces cerevisae and/or cell-cultured cells like hepatocytes and/or cardiomyocytes as shown in step 30 of FIG. 2. A dilute emulsion of a lipophilic culture medium can also be used to test for lipophilic antioxidants.

Cells of the medium are first sedimented using a centrifugation process and washed to remove any extracellular reactive oxygen species (ROS) that may be present in the supernatant of the yeast suspension, as shown in steps 31 and 32 of FIG. 2. The cells are then suspended in a sodium phosphate buffer solution to create an environment that is not harsh to yeast cells, as shown in step 33 of FIG. 2. Next, the cells are incubated with a 2-7 Dichlorofluorescin (H₂DCFDA) probe, known in the art as a fluorescent dye, marketed by Molecular Probes, Eugene Ore. as a D-399 compound, as shown in step 34 of FIG. 2.

Intracellularly, the H₂DCFDA probe penetrates the plasma membrane and the diacetate form of the dye (H₂DCF-DA) is readily transported across the cellular membrane into the cell interior.

Once inside the cell, non-specific esterases hydrolyze the compound yielding products that are more polar that are trapped within the cell.

Intracellular esterases can then cleave the acetate groups producing a non-fluorescent form of the dye (H₂DCF), as shown in step 35 of FIG. 2, that becomes fluorescent upon encounter with various reactive oxygen species (ROS), as shown in step 36 of FIG. 2.

The acetate groups can also be cleaved chemically with a dilute solution of sodium hydroxide to produce the H₂DCF form of the dye. This form could then be introduced to the extracellular medium.

The diacetate free form of the dye can thus be used to detect both intracellular and extracellular ROS without having to know whether or not the dye is actively taken into the cell species.

The cells can further be incubated with an oxidation catalyst promoter such as H₂O₂, peroxidase, transition metals, hydroxides or superoxides, to increase oxidative activity. The promoter can be used as a control to show that the probe is converting from H₂DCF to DCF fluorescent.

A promoter such as H₂O₂, for example, can be used with cell cultures and horseradish peroxidase (HRP) can be used with both yeast cells and cell cultures.

The generation of ROS in excess by peroxidase, for example, assures complete availability of free radicals in a form that actively binds the dye. The excess ROS also allows for increased sensitivity of the fluorescent response.

The fluorescent form of the dye is then read fluorometrically, using an optical fiber sensor, as shown in step 37 of FIG. 2 and the progress of the oxidation process is graphed kinetically as indicated in step 38 of FIG. 2.

Steps 30-36 of FIG. 2 form a control group as shown by block 45 of FIG. 2. In the control group cells, there is a gradual increase in the presence of DCF as the original compound becomes oxidized. This process is a measure of the internal ROS produced through normal metabolism.

Next, the control group 45 is incubated with an antioxidant sample comprising a food-based source of a key phytonutrient or vitamin with antioxidant capabilities such as wheat germ oil (a source of vitamin E), as shown in step 40 of FIG. 2.

The antioxidant samples may include fresh whole foods, dried whole foods that are manufactured in supplement form, vitamins and phytonutrients synthesized and/or isolated, and pre-formulated nutritional supplements that are designed to demonstrate antioxidant activity when administered orally.

The free radical scavenging activity of the antioxidants is then measured fluorimetrically, using an optical fiber sensor, as shown in step 41 of FIG. 2 where a relative decrease in the presence of DCF is observed as the antioxidants function to scavenge the reactive oxygen species.

Next, the fluoroscopic measurements are graphed and assayed as shown in step 42 of FIG. 2 to compare the relative antioxidant capacity of the product formulation.

An antioxidant can be characterized by its scavenging properties, with the inhibition percentage and inhibition over time parameters measured, for example, with a microplate style fluorometer.

By setting up a kinetic study and using an area under the curve (AUC) quantitation method, as is known in the art, the parameters can be combined into a single measurement to compare the antioxidant capacity of different formulations. In addition, a water-soluble α-tocopherol analog calibrator such as Trolox, for example, can be used with the OASP to standardize readings with relative fluorescent units (RFU's) obtained with calibration of the salt form of the probe (DCF). The correlation is based on a prepared standard curve that has proven to be linear (R²>0.99) over three orders of magnitude. Concentrations are detectable in the pM range.

Measurements are performed on a blank sample, consisting of the probe and buffer, to form a baseline that can be subtracted from measurements made on the antioxidant sample to calculate the metabolic ROS.

The OASP probe of the invention can also measure peroxyl radicals and has been shown to detect hydrogen peroxide, organic hydroperoxides, nitric oxide, and peroxynitrite. In addition, the probe has shown to be responsive to hydroxyls and transition metal oxidative damage, which is a good measure of overall oxidative stress.

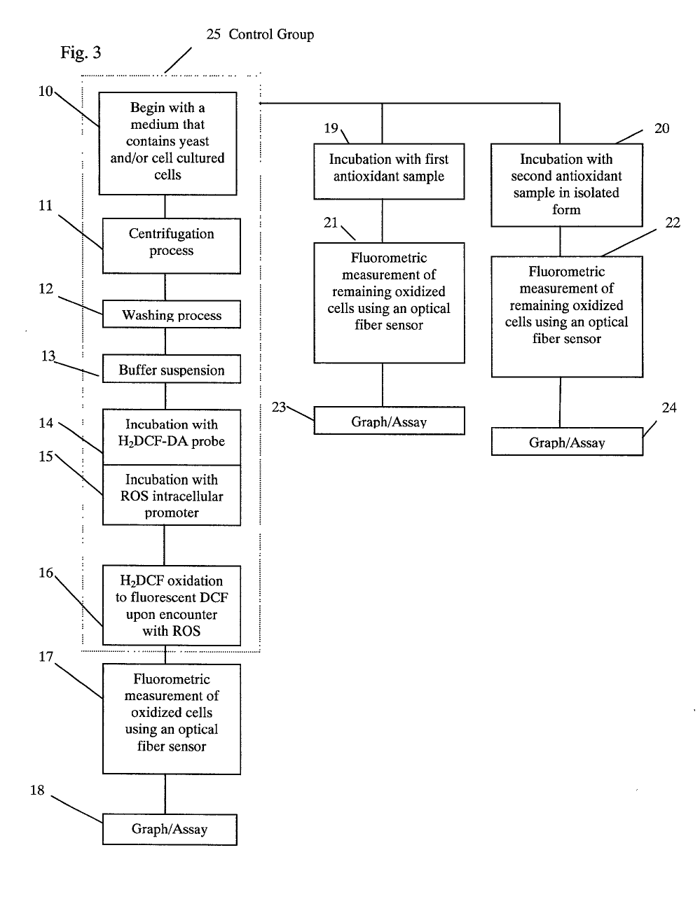

An OASP for comparing the efficiency of a food-based antioxidant to an isolated form of the antioxidant is shown in FIG. 3.

With reference to FIG. 3, the medium used in the OASP contains live yeast cells such as sacchromyces cerevisae and/or cell-cultured cells like hepatocytes and/or cardiomyocytes as shown in step 10 of FIG. 3. A dilute emulsion of a lipophilic culture medium can also be used to test for lipophilic antioxidants.

Cells of the medium are first sedimented using a centrifugation process and washed as shown in steps 11 and 12 of FIG. 3 to remove extracellular ROS. The cells are then suspended in a buffer solution as shown in step 13 of FIG. 3. Next, the cells are incubated with a 2-7 Dichlorodihydrofluorescein (H₂DCFDA) probe, as shown in step 14 of FIG. 3.

The cells can be further incubated with an oxidation catalyst promoter, such as H₂O₂, transition metals, hydroxides or superoxides, or preferably peroxidase, to increase oxidative activity, as shown in step 15 of FIG. 3. A promoter such as horseradish peroxidase (HRP), for example, can be used with both yeast cells and cell cultures, and H₂O₂, for example, can be used with cell cultures.

The H₂DCF oxidation to fluorescent DCF, upon encounter with ROS, is shown in step 16 of FIG. 3.

The fluorescent moiety of the cells is then read fluorometrically, using an optical fiber sensor, as shown in step 17 of FIG. 3 and the progress of the oxidation process is graphed as indicated in step 18 of FIG. 3.

Steps 10-16 of FIG. 3 form a control group as shown by block 25 of FIG. 3. In the control group cells, there is a gradual increase in the presence of DCF as the original compound becomes oxidized. Next, the control group 25 is incubated with an antioxidant sample comprising a food-based source of a key phytonutrient or vitamin with antioxidant capabilities such as wheat germ oil (a source of vitamin E), as shown in step 19 of FIG. 3

A second portion of the control group 25 is incubated with the same phytonutrient or antioxidant vitamin, in isolated form, such as Trolox (an isolated form of vitamin E proven to have antioxidant activity), for example, as shown in step 20 of FIG. 3.

The antioxidant samples may include fresh whole foods, dried whole foods that are manufactured in supplement form, vitamins and phytonutrients synthesized and/or isolated, and pre-formulated nutritional supplements that are designed for the purpose of demonstrating antioxidant activity when administered orally.

The free radical scavenging activity of the antioxidants is then measured fluorimetrically, using an optical fiber sensor, as shown in steps 21 and 22 of FIG. 3 where a relative decrease in the presence of DCF is observed as the antioxidants function to scavenge the reactive oxygen species.

Next, the fluoroscopic measurements are graphed and assayed as shown in steps 23 and 24 of FIG. 3 to compare the relative antioxidant capacity of the product formulations.

The findings of the antioxidant capacity assay of the OASP can also be utilized to evaluate processes in a living human biological system.

As one ingests a functional food supplement, it passes through a gastrointestinal microbial ecosystem that affects how the product is broken down and metabolized and ultimately affects the product's influence on intracellular antioxidant activity.

By introducing the product (a functional food-based antioxidant) to select steps in the digestive process, and drawing out the sample from a select vessel, representative of a specific step in the digestive process, a researcher can determine which segment in the gastrointestinal microbial ecosystem provides the optimal degree of intracellular free radical scavenging action by the metabolites produced from the AO (antioxidant) supplement.

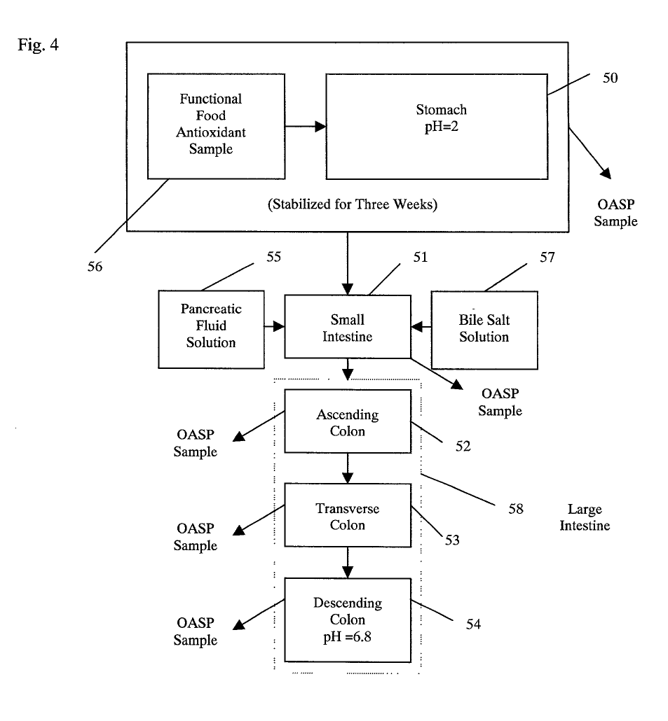

A biological sampling system using the OASP of the invention is shown in FIG. 4. The system is broken down into five vessels representing five segments of the gastrointestinal (GI) tract: stomach 50, small intestine 51, ascending colon 52, transverse colon 53, and descending colon 54. All vessels are maintained at 98.6 F (body temperature). The vessels contain individual ingredients that serve to mimic the environment in a corresponding segment in the GI tract. The pH is also controlled and varies between vessels depending upon the pH range that is considered to be normal for that portion of the GI tract.

The first vessel 50 is maintained at a pH of 2, for example, simulating the acidic environment of the stomach, while the descending colon segment 54 is maintained at a pH of 6.8 mimicking the alkaline environment of that portion of the GI tract.

Pancreatic fluid 55 and a bile salt solution 57 are added in appropriate vessels to the small intestine vessel 51 to simulate the small intestine environment 51.

A functional food antioxidant sample 56 is added to the first vessel 50 and stabilized over a three-week period. The resulting solution is then pumped into the second vessel 51 where a pancreatic fluid solution 55 and a bile salt solution 57 are added to mimic the environment and pH of the small intestine 51.

A sample can be drawn from any one of these vessels and then introduced into the OASP methodology. The effects of the resultant sample on intracellular antioxidant capacity can then be determined depending on where it passes in the GI tract. This same process can be used with the other vessels that represent the three segments of the large intestine 58.

Depending upon which vessel the sample is drawn from, that solution's relative intracellular effect on free radicals can be measured to determine, for example, if the antioxidant product should be enteric coated to by-pass the effects of the acidic stomach environment.

The OASP is a cost-effective and timesaving method of performing an assay, as the only major equipment required is a fluorometer and a centrifuge water bath. The assay can be performed in a microplate format fluorometer and be completed in as little as three hours.

In summary, the OASP of the invention can be used with virtually any cellular system in an in-vitro or in-vivo environment to objectively measure the effectiveness of a food-based antioxidant supplement or even the whole food compared to that of an isolated antioxidant. Measurement with the OASP is not only a measure of the antioxidant capacity of a food or supplement however, it is a measure of the protective effects provided to a cellular system in much the same way as would occur in our bodies.

Although the various features of novelty that characterize the invention have been described in terms of certain preferred embodiments, other embodiments will become apparent to those of ordinary skill in the art, in an objective view of the disclosure herein. Accordingly, the present invention is not intended to be limited by the recitation of the preferred embodiments, but is instead intended to be defined solely by reference to the appended claims.

What is claimed is:

1. An optical antioxidant sensing process for measuring the free radical scavenging efficiency of a nutritional formulation when encountering reactive oxygen radical species in a medium comprising the steps of:
introducing an organic dye reagent that reacts with oxygen radicals to said medium to chemically tag said oxygen radicals in said medium;
detecting and measuring the population of said tagged oxygen radicals using an optical fiber sensor;
introducing a nutritional formulation with antioxidant properties to said medium;
detecting and measuring the relative population of said tagged oxygen radicals in said medium using said optical fiber sensor;
calculating the free radical scavenging efficiency of said nutritional formulation using said oxygen radical population measurements; and
assaying the free radical scavenging effectiveness of the nutritional formulation.

2. The optical antioxidant sensing process of claim 1 wherein said organic dye reagent has a chemical composition that diffuses through a cell membrane.

3. The optical antioxidant sensing process of claim 1 where said organic dye reagent is 2-7 Dichlorofluorescin (H₂DCFDA).

4. An optical antioxidant sensing process for measuring the free radical scavenging efficiency of a nutritional formulation when encountering reactive oxygen radical species in a medium comprising the steps of:
introducing an organic dye reagent that reacts with oxygen radicals to said medium to chemically tag said oxygen radicals in said medium;
introducing an oxygen catalyst promoter to said medium to increase oxidative activity;
detecting and measuring the population of said tagged oxygen radicals using an optical fiber sensor;
introducing a nutritional formulation with antioxidant properties to said medium;
detecting and measuring the relative population of said tagged oxygen radicals in said medium using said optical fiber sensor;
calculating the free radical scavenging efficiency of said nutritional formulation using said oxygen radical population measurements; and
assaying the free radical scavenging effectiveness of the nutritional formulation.

5. The optical antioxidant sensing process of claim 4 wherein said organic dye reagent has a chemical composition that diffuses through a cell membrane.

6. The optical antioxidant sensing process of claim 4 where said organic dye reagent is 2-7 Dichlorofluorescin (H₂DCFDA).

7. The optical antioxidant sensing process of claim 4 wherein said oxygen catalyst promoter is from the group consisting of H₂O₂, peroxidase, transition metals, hydroxides and superoxides.

8. The optical antioxidant sensing process of claim 4 wherein said oxygen catalyst promoter is horseradish peroxidase.

9. An optical antioxidant sensing process to measure the free radical scavenging efficiency of nutritional formulations comprising:
forming a control group including a medium that contains tagged fluorescent oxygen radicals;
incubating a first portion of said control group with a first sample of a nutritional formulation having antioxidant properties;
incubating a second portion of said control group with a second sample of said nutritional formulation having antioxidant properties in isolated form;
measuring the free radical scavenging activity of said first sample in said first portion of said control group using an optical fiber sensor;
measuring the free radical scavenging activity of said second sample in said second portion of said control group using said optical fiber sensor; and
assaying the antioxidant capacity of said first and said second samples.

10. The optical antioxidant sensing process of claim 9 wherein said first sample comprises a food-based source of a key phytonutrient with antioxidant capabilities.

11. The optical antioxidant sensing process of claim 9 wherein said first sample comprises a vitamin with antioxidant capabilities such as wheat germ oil.

12. The optical antioxidant sensing process of claim 9 wherein said second sample comprises a key phytonutrient with antioxidant capabilities in isolated form.

13. The optical antioxidant sensing process of claim 9 wherein said second sample comprises a vitamin with antioxidant capabilities in isolated form.

14. The optical antioxidant sensing process of claim 9 wherein said second sample comprises wheat germ oil.

15. The optical antioxidant sensing process of claim 9 wherein said second antioxidant sample is an isolated form of vitamin E proven to have antioxidant activity.

16. The optical antioxidant sensing process of claim 9 wherein said second antioxidant sample is Trolox.

17. A process for measuring antioxidant activity in an in-vitro model mimicking the gastrointestinal tract comprising the steps of:
introducing a functional food-based antioxidant sample to a first vessel containing ingredients that mimic the environment in a stomach segment of said gastrointestinal tract;
pumping the resultant solution into a second vessel containing ingredients that mimic the environment in a small intestine segment of said gastrointestinal tract;
introducing a pancreatic fluid solution to said second vessel to further mimic the environment of said small intestine segment;
introducing a bile salt solution to said second vessel to further mimic the environment in said small intestine segment;
pumping the resultant solution into a third vessel containing ingredients that mimic the environment in a large intestine segment of said gastrointestinal tract; and
assaying solutions from said vessels using the optical antioxidant sampling process of the invention to determine the solution's relative intracellular effects on free radicals in said gastrointestinal tract.
